(19) United States
(12) Patent Application Publication
(10) Pub. No.: US 2023/0230685 A1
(43) Pub. Date: Jul. 20, 2023

(54) Intelligent Matching Of Patients With Care Workers

(71) Applicant: A2B Directcare Inc., Toronto (CA)

(72) Inventors: Chen Little, Toronto (CA); Amandeep Sandhu, Toronto (CA)

(73) Assignee: A2B Directcare Inc., Toronto (CA)

(21) Appl. No.: 18/096,550

(22) Filed: Jan. 12, 2023

Related U.S. Application Data
(60) Provisional application No. 63/298,662, filed on Jan. 12, 2022.

(51) Int. Cl. G16H 40/20; G16H 10/60

(52) U.S. Cl. CPC G16H 40/20; G16H 10/60

(57) ABSTRACT

A system for a care provider recommendation including: a processor of a recommendation server connected to a user device over a network and configured to host a machine learning module; a memory on which are stored machine-readable instructions that when executed by the processor, cause the processor to: receive a care provider request including user profile data from the user device; parse the care provider request to derive a plurality of features from the user profile data; query an aggregated care providers' database to retrieve historical care providers' matching data based on the user profile data; generate a feature vector based on the plurality of features and the historical care providers' matching data; and provide the feature vector to the ML module for generating a predictive model configured to output one or more care provider selection parameter for generating a care provider recommendation responsive to the care provider request.

### CROSS-REFERENCES TO RELATED APPLICATIONS

This application claims priority from U.S. Provisional Application Ser. No. 63/298,662 filed Jan. 23, 2022 which is hereby incorporated herein by reference in its entirety.

### TECHNICAL FIELD

This application generally relates to matching of clients to service providers, and more particularly, to an intelligent matching AI-based process facilitated by an automated assessment of the clinical, social, and environmental contexts of clients/patients and community care workers.

### BACKGROUND

Health delivery providers typically address the issue of matching patients with providers by reviewing their rolodex of care workers, and determining the best fit based on availability of schedules. Other health tech providers have built online marketplaces that primarily consider proximity, ratings, and user activity. The existing system and applications do not provide for personalized home care experience. The existing matching protocol do not consider factors like specialization, language, interests, hobbies, rating based on past experience evaluated by hours worked (not only by client/patient feedback), life experiences, health, response rates of care workers, and cultural understanding.

While existing health delivery providers often claim that they consider the above factors, however in reality most often cannot match the patient with a provide based on the factors. All matching is performed manually and is inefficient. Furthermore, existing providers and marketplaces do not consider factors such as care circle composition beyond medical professionals, geography and languages spoken of care circle members, skills and expertise of family members, and a cultural understanding of the patient/family as well as their personal values and attitudes towards care when manually matching a patient with a potential provider.

Managing clients often comes with the roles of various kinds of service providers (care coordinators, care providers, families, and other organizations involved in a patient's journey). For this reason, it is important to manage and care for the patient responsibly and efficiently. With incoming streams of information from places such as, but not limited to telemedicine, diagnostics tests, doctors' notes, nurses' notes, patients' notes, care givers' notes, families etc., often care providers (which in some instances can be but are not limited to family members, personal support workers, nurses and/or doctors) can become overwhelmed by the volume of information being offered to them.

For this reason, when managing caring for patients, it is also important to monitor the patient's needs thoroughly and intelligently deliver the necessary critical information required for making a decision, efficiently. By simply delivering all the information to a care provider, it often overloads care providers with information. For this reason, it is important to deliver an intelligent data flow. By using artificial intelligence (AI) to identify the importance of data passed onto a service provider which would be strategically beneficial for their care, a system can be devised to better manage the care of a patient. The conventional protocols fail to utilize combinations of multiple factors in producing the most accurate family-care worker matches, while optimizing for a low frontline turnover.

As such, what is needed is an effective solution for intelligent matching process for matching patients with care providers facilitated by an automated assessment of the clinical, social, and environmental contexts of clients/patients and community care workers.

### SUMMARY

One example embodiment provides a system for a care provider recommendation including: a processor of a recommendation server connected to a user device over a network and configured to host a machine learning module; a memory on which are stored machine-readable instructions that when executed by the processor, cause the processor to: receive a care provider request including user profile data from the user device; parse the care provider request to derive a plurality of features from the user profile data; query an aggregated care providers' database to retrieve historical care providers' matching data based on the user profile data; generate a feature vector based on the plurality of features and the historical care providers' matching data; and provide the feature vector to the ML module for generating a predictive model configured to output one or more care provider selection parameter for generating a care provider recommendation responsive to the care provider request.

Another example embodiment provides a method that includes one or more of: receiving a care provider request including user profile data from the user device; parsing the care provider request to derive a plurality of features from the user profile data; querying an aggregated care providers' database to retrieve historical care providers' matching data based on the user profile data; generating a feature vector based on the plurality of features and the historical care providers' matching data; and providing the feature vector to the ML module for generating a predictive model configured to output one or more care provider selection parameter for generating a care provider recommendation responsive to the care provider request.

Yet another disclosed embodiment provides a non-transitory computer readable medium having instructions, that when read by a processor, cause the processor to perform: receiving a care provider request including user profile data from the user device; parsing the care provider request to derive a plurality of features from the user profile data; querying an aggregated care providers' database to retrieve historical care providers' matching data based on the user profile data; generating a feature vector based on the plurality of features and the historical care providers' matching data; and providing the feature vector to the ML module for generating a predictive model configured to output one or more care provider selection parameter for generating a care provider recommendation responsive to the care provider request.

### BRIEF DESCRIPTION OF THE DRAWINGS

FIG. 1 illustrates a diagram of a system for intelligent matching process for matching patients with care providers, according to example embodiments;

FIG. 1A illustrates a network diagram of a system for an intelligent AI-based automated recommendation for a selection of a care provider consistent with the present disclosure;

FIG. 1B illustrates a network diagram of a system for an intelligent AI-based automated recommendation for selection of a care provider using a blockchain consistent with the present disclosure;

FIG. 2 illustrates a network diagram of a system including detailed features of a recommendation server (RS) node consistent with the present disclosure;

FIG. 3A illustrates a flowchart of a method for an intelligent AI-based automated recommendation for selection of a care provider consistent with the present disclosure;

FIG. 3B illustrates a further flow chart of a method for the intelligent AI-based automated recommendation for selection of the care provider consistent with the present disclosure;

FIG. 4 illustrates deployment of a machine learning model for prediction of care provider selection parameters using blockchain assets consistent with the present disclosure;

FIG. 5 illustrates a block diagram of a system including a computing device for performing the method of FIGS. 3A and 3B.

### DETAILED DESCRIPTION

It will be readily understood that the instant components, as generally described and illustrated in the figures herein, may be arranged and designed in a wide variety of different configurations. Thus, the following detailed description of the embodiments of at least one of a method, apparatus, non-transitory computer readable medium and system, as represented in the attached figures, is not intended to limit the scope of the application as claimed but is merely representative of selected embodiments.

The instant features, structures, or characteristics as described throughout this specification may be combined or removed in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment. Thus, appearances of the phrases “example embodiments”, “in some embodiments”, “in other embodiments”, or other similar language, throughout this specification do not necessarily all refer to the same group of embodiments, and the described features, structures, or characteristics may be combined or removed in any suitable manner in one or more embodiments. Further, in the diagrams, any connection between elements can permit one-way and/or two-way communication even if the depicted connection is a one-way or two-way arrow. Also, any device depicted in the drawings can be a different device. For example, if a mobile device is shown sending information, a wired device could also be used to send the information.

In addition, while the term “message” may have been used in the description of embodiments, the application may be applied to many types of networks and data. Furthermore, while certain types of connections, messages, and signaling may be depicted in exemplary embodiments, the application is not limited to a certain type of connection, message, and signaling.

Example embodiments provide methods, systems, components, non-transitory computer readable media, devices, and/or networks, which provide for intelligent matching process for matching patients with care providers facilitated by an automated assessment of the clinical, social, and environmental contexts of clients/patients and community care workers.

According to one disclosed embodiment, the exemplary matching system provides clients/patients with an unparalleled personalized home care experience. The exemplary AI-based machine learning (ML) enhanced matching protocol considers factors like proximity, specialization, language, and cultural understanding. This protocol was generated based on mixed-method research studies and data analysis from tens of thousands of successful care appointments. The purpose of the matching protocol is to utilize combinations of numerous factors in producing the most successful and “stickiest” family-care worker matches, while optimizing for low frontline turnover.

The matching protocol may ingest and may process the following parameters:
Care circle composition—e.g., client/patient, family caregivers, other health care providers;
Care arrangements—e.g., frequency of care needs, geography of care circle members;
Care processes—e.g., care needs, schedules, decision-making related arrangements;
Resources of care worker—e.g., time, knowledge, expertise, skills; and
Care worker performance—e.g., care worker reviews from previous client engagements.
Care worker experience with patients—e.g., care worker hours worked with previous client engagements.
Care worker experience at other workplaces—e.g., the type of work and experience at other workplaces. Including but not limited to the types of roles at particular jobs.

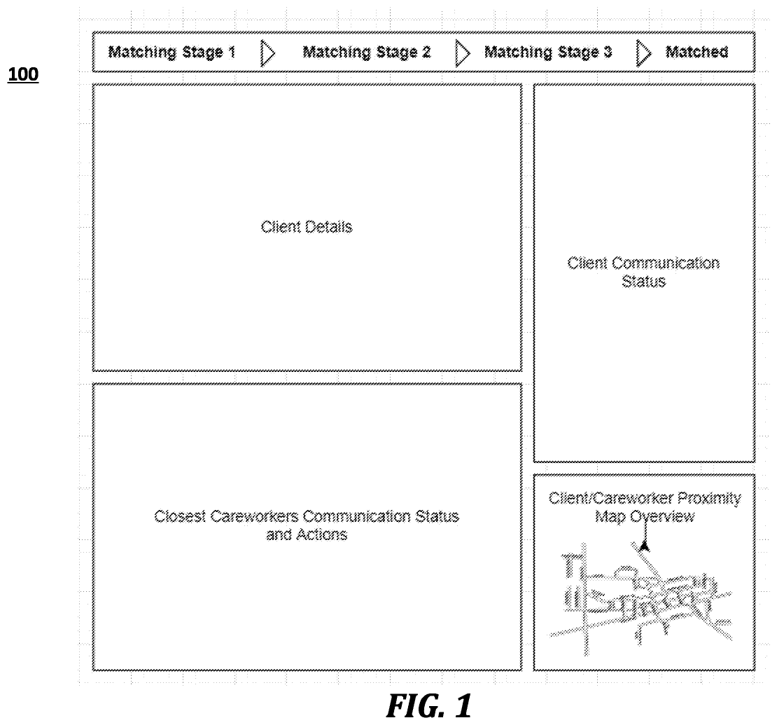

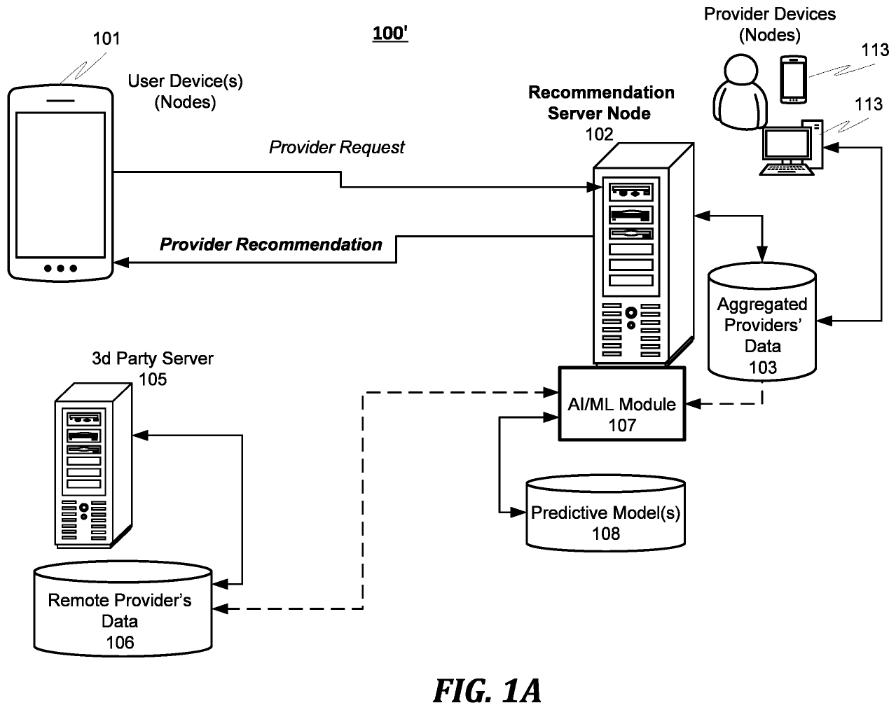

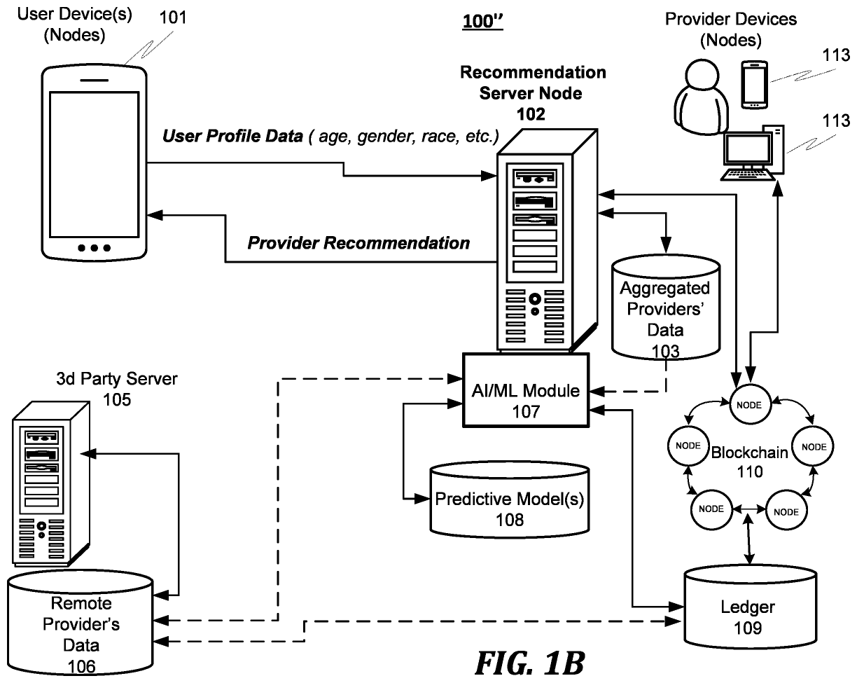

FIG. 1 illustrates a diagram of a system for AI-based intelligent matching process for matching patients with care providers, according to example embodiments.

Referring to FIG. 1, the system architecture 100 is designed to permit the fusion of multiple data and communication methods. Data sources and communication methods may be removed or added to the central operations management system to facilitate the matching technology (i.e., matching stages) based on changing data contexts and/or communication preferences.

One embodiment may center around the client/patient file to facilitate automated matching based on the clinical, social, and environmental contexts and clients/patients and care providers/workers. The matching portal staff may utilize the client file view to assess the status of the automated matching and may perform actions to support the process.

Unlike traditional marketplace algorithms, the exemplary technology strives to optimize the matching process while eliminating the need to reach out to a large number of care workers to complete a successful match. The disclosed embodiments of the proposed architecture allow for new, updated data and/or communication sources to be plugged in and integrate into the client file/details 111. For example, one embodiment may permit the integration of a new communications chat application to be added, or an additional client context to be added as new client data becomes available.

The modular design of the central operations management system fuses the data from clients and care providers and follows a plugin architecture for the matching technology to utilize dynamic data sources to best match the client and the care provider. The additional matching parameters may include:

Care relationships—e.g., nature and quality of relationships between care circle members;

Common interests between the care worker and the client—e.g., hobbies and interests;

Care worker physical capabilities—e.g., sports/recreational activities they partake in;

Formal education of the care worker;

Perspectives and attitudes toward care—e.g., personal and collective values; and

Resources of patient/family—e.g., financial, time, knowledge, expertise, skills.

In one disclosed embodiment, the AI/ML technology may be combined with a blockchain technology for secure use of user personal medical and financial data. The disclosed embodiment may produce a detailed care provider score for the current requesting user (on and off line) based on the user profile data settings. In one embodiment, the care providing authorities may be connected to the recommendation server (RS) over a blockchain network to achieve a consensus prior to executing a transaction to release the new care provider recommendations to the requesting user.

FIG. 1A illustrates a network diagram of a system for an intelligent AI-based automated recommendation for a care provider consistent with the present disclosure.

Referring to FIG. 1A, the example network 100′ includes a recommendation server (RS) node 102 connected to a 3d-pary server node(s) 105 over a network. The 3d-pary server node(s) 105 may be a cloud server. The RS node 102 is configured to host an AI/ML module 107. The RS node 102 may receive a care provider request data from a user device 101 that may be a smartphone, a tablet, notebook, etc. Note that the user device 101 may provide an IP address or its geo-location. The provider request may include user profile data (e.g., name, location, language, age, gender, race, hobbies, etc.). The RS node 102 may query a providers' aggregated database 103 for the provider profiles data and historical matching data based on the user profile data received from the requesting user of the device 101. In one embodiment, the provider devices—i.e., devices (or network nodes) 113 may provide constant updates of the data reflecting the current provider offers to the aggregated database 103. The RS node 102 may also acquire relevant provider matching data 106 from a remote database 106 residing on a 3d-party cloud server(s) 105. The 3d-party cloud server(s) 105 may be onboarded to the network 100′ based on a prior agreement and consensus. The previous providers' matching data 106 may be collected from the users of the same type (e.g., age, gender, race, age, etc.) who used the providers from other organizations that are located within a certain range of the current location of the requesting user of the device 101.

The RS node 102 may generate a feature vector data based on the user profile data (i.e., all user profile parameters, doctors'/nurses' and/or family recommendations) entered via an interface on the user device 101 prior to making a care provider request and the collected historical data (i.e., pre-stored matching data 103 and 3d-party matching data 106). The RS node 102 may ingest the feature vector data into an AI/ML module 107. The AI/ML module 107 may generate a predictive model(s) 108 based on the feature vector data to predict care provider selection parameters for automatically generating a recommendation to the user of the device 101 that had initiated the care provider request. The parameters may be provided to the user device 101 in a form of textual or imagery recommendation (i.e., the image displaying the picture(s) and rankings of the provider(s) recommended to the user that can be easily selected on the user device 101 screen for a quick selection and service payment transaction).

Client data may be submitted by the client (i.e., the device 101) through website submissions and SMS-based exchanges. The data is then submitted to the central operations system (i.e., the RS node 102) for processing. Upon approval of the client, the matching algorithms may be executed on the AI/ML module 107 to determine the best care worker/provider match for the client based on:

a. Care circle composition—informal care (e.g., family caregivers);

b. Care circle composition—formal health care providers;

c. Geographic location of care circle members;

d. Geographic location of care worker;

e. Gender of client;

f. Gender of care worker;

g. Languages spoken in client household;

h. Languages spoken by care worker and degree of fluency;

i. Types of care needs;

j. Training/specialization required of care worker based on care needs;

k. Frequency of care needs;

l. Time availability of care worker;

m. Years of experience of care worker;

n. Access to a car for transportation of a client;

o. Phone access of a client;

p. Phone access of a care worker;

q. Care worker reviews from previous client engagements;

r. Care worker interests and hobbies;

s. Client interests and hobbies;

t. Training the algorithm/matching metrics based on the response times over time;

u. Training the algorithm/matching metrics based on the response rate of responses from the care worker;

u. Care worker general work experience;

v. Clients and care worker notes regarding the client's health to help determine ongoing care/protocol; and

w. Clients medical devices to monitor the ongoing health of the patient to ensure strategic monitoring.

The matching algorithm may rank each care provider according to the score based on the above criteria and may display the care providers to the user of the device 101 according to the score by descending order. Upon a confirmation of a new client file, one embodiment of the automated match communication process may take place via SMS messaging to best matched care providers in order of the score. Blocks of best matched care providers may be sent an SMS, one block of care providers at a time, with an elapsed amount of time given to provide the opportunity for care providers to respond before sending an SMS to the next block. When a number of care providers indicate interest in being matched with the client, the automated match communication process exits and the care providers who have been matched and indicate interest are flagged for associate platform support staff.

Another embodiment, may center around the assessment of the patient and/or manage care for a patient better. By gathering the incoming data and labeling the patients, the RS node 102 may calculate risk factors of the patient to determine efficient patient management without overwhelming the care provider or any other parties helping to manage the patients overall care plan. Additionally, analyzing this data prior to it being shared with other care providers and/or organizations can help alleviate the pressures of handling too much data, which risks overwhelming the care provider.

By using incoming information from places such as, but not limited to, telemedicine, diagnostics tests, doctors' notes, nurses' notes, patients' notes, care givers' notes, families etc., evaluation of care capacity in the home and/or by their surroundings, and/or insurance information, we assess the importance and of any incoming information from any of the following. The RS node 102 may then automatically use a machine learning algorithm which assess the data and delivers either the data necessary for managing the patient which was labeled as flagged data, or the unlabeled data which is deemed as non-alerts. This way, care coordinators and/or any organization managing the health of the patient and/or recommending treatment are not overwhelmed by the incoming data and can manage the patient based on the issues. This enables action based on data which is deemed important after automated review.

All communication via SMS messages between the client/care worker and the automated system are saved in the central operations management system (e.g., in the database 103) that includes the text of the message, as well as identifying information to allow retrieval and viewing of the communication chain by search criteria including client file (containing the user profile data), time and date.

FIG. 1B illustrates a network diagram of a system for an intelligent AI-based automated recommendation for a selection of a care provider using a blockchain consistent with the present disclosure.

Referring to FIG. 1B, the example network 100″ includes a recommendation server (RS) node 102 connected to a 3d-pary server node(s) 105 over a network. The 3d-pary server node(s) 105 may be a cloud server. The RS node 102 is configured to host an AI/ML module 107. The RS node 102 may receive a care provider request data from a user device 101 that may be a smartphone, a tablet, notebook, etc. Note that the user device 101 may provide an IP address or its geo-location. The provider request may include user profile data (e.g., name, location, language, age, gender, race, hobbies, etc.). The RS node 102 may query a providers' aggregated database 103 for the provider profiles data and historical matching data based on the user profile data received from the requesting user of the device 101. In one embodiment, the provider devices—i.e., devices (or network nodes) 113 may provide constant updates of the data reflecting the current provider offers to the aggregated database 103. The RS node 102 may also acquire relevant provider matching data 106 from a remote database 106 residing on a 3d-party cloud server(s) 105. The 3d-party cloud server(s) 105 may be onboarded to the network 100′ based on a prior agreement and consensus. The previous providers' matching data 106 may be collected from the users of the same type (e.g., age, gender, race, age, etc.) who used the providers from other organizations that are located within a certain range of the current location of the requesting user of the device 101.

The RS node 102 may generate a feature vector data based on the user profile data (i.e., all user profile parameters, doctors'/nurses' and/or family recommendations) entered via an interface on the user device 101 prior to making a care provider request and the collected historical data (i.e., pre-stored matching data 103 and 3d-party matching data 106). The RS node 102 may ingest the feature vector data into an AI/ML module 107. The AI/ML module 107 may generate a predictive model(s) 108 based on the feature vector data to predict care provider selection parameters for automatically generating a recommendation to the user of the device 101 that had initiated the care provider request. The parameters may be provided to the user device 101 in a form of textual or imagery recommendation (i.e., the image displaying the picture(s) and rankings of the provider(s) recommended to the user that can be easily selected on the user device 101 screen for a quick selection and service payment transaction).

In one embodiment, the RS node 102 may receive the predicted care provider recommendation parameters from a permissioned blockchain 110 ledger 109 based on a consensus from the provider devices 113. Additionally, confidential historical care provider matching information and previous care provider recommendations may also be acquired from the permissioned blockchain 110. The newly acquired user profile data with corresponding predicted care provider recommendation parameters data may be also recorded on the ledger 109 of the blockchain 110 so it can be used as training data for the predictive model(s) 108. In this implementation the RS node 102, the 3d-party cloud server 105 and the care provider devices 113 may serve as blockchain 110 peer nodes. In one embodiment, aggregated data 103 and remote data 106 may be duplicated on the blockchain ledger 109 for a higher security of storage.

The AI/ML module 107 may generate a predictive model(s) 108 to predict the care provider recommendation parameters for the user device 101 in response to the specific relevant pre-stored care provider matching data acquired from the blockchain 110. This way, the current care provider recommendation parameters may be predicted based not only on the live user profile data, but also based on the previously collected historical care provider matching data and 3d-party server care provider matching data associated with the given user (or users of the same type based on age, gender, race, locations, etc.).

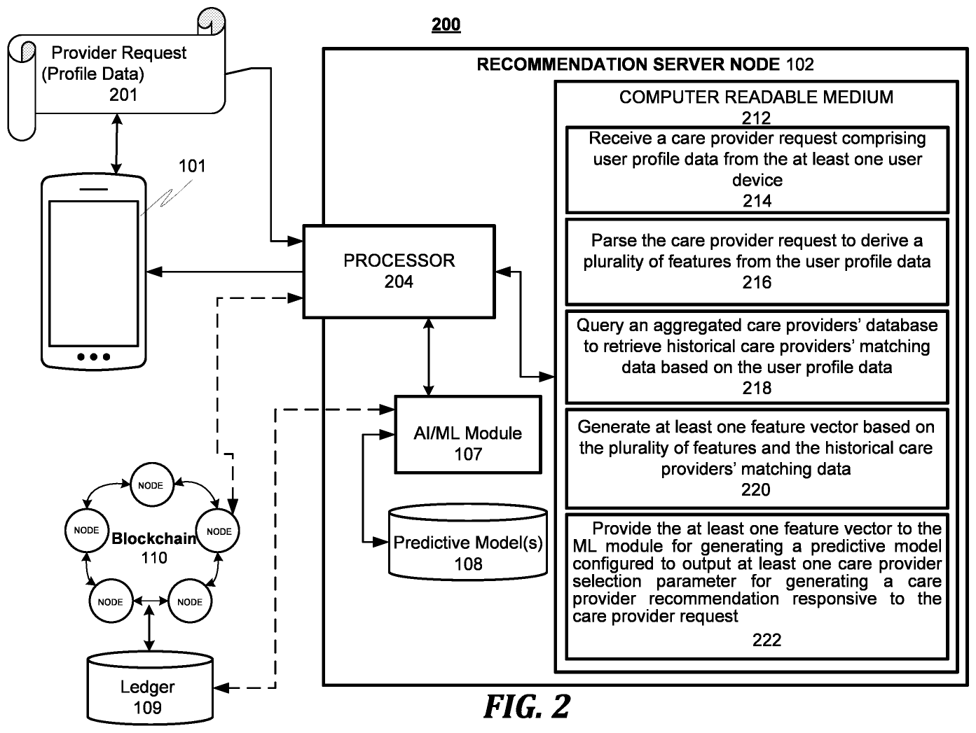

FIG. 2 illustrates a network diagram of a system including detailed features of a recommendation server (RS) node consistent with the present disclosure.

Referring to FIG. 2, the example network 200 includes the RS node 102 connected to a user device 101 over a network (LAN or wireless). The RS node 102 may be configured to host an AI/ML module 107. As discussed above with reference to FIGS. 1A-B, the RS node 102 may receive a care provider request including user profile data along with and pre-stored historical care provider recommendation/matching data retrieved from local and remote databases (not shown). As discussed above, the pre-stored care provider recommendation data may be retrieved from the ledger 109 of the blockchain 110.

The AI/ML module 107 may generate a predictive model(s) 108 based on the care provider request and care provider matching-related data provided by the RS node 102. As discussed above, the AI/ML module 107 may provide predictive outputs data in a form of care provider recommendation parameters for generation of an automatic recommendation to the requesting user of the user device 101. The RS node 102 may process the predictive outputs data received from the AI/ML module 107 to generate a care provider recommendation in either textual or visual form (i.e., a clickable image of the recommended user providers).

In one embodiment, the RS node 102 may acquire user profile data periodically in order to check if the user of the device 101 has added or removed some of his user profile parameters or the user has changed his/her location. The user profile data may be automatically acquired by the RS 102 by scanning the user device 101 based on the user's consensus. In another embodiment, the RS node 102 may continually monitor user profile data and may compare this data against current care provider offers received by the RS node 102. For example, if a new care provider or a new type of service is introduced, the RS node 102 may automatically run the new care provider information through the AI/ML module 107 based on the current user profile and may produce a recommendation to engage or request the new care provider. As another non-limiting example, if terms and service conditions of the current care provider associated by the service agreement with the user profile change, the RS node 102 may recommend to drop this care provide and may recommend a more suitable one.

While this example describes in detail only one RS node 102, multiple such nodes may be connected to the network and to the blockchain 110. It should be understood that the RS node 102 may include additional components and that some of the components described herein may be removed and/or modified without departing from a scope of the RS node 102 disclosed herein. The RS node 102 may be a computing device or a server computer, or the like, and may include a processor 204, which may be a semiconductor-based microprocessor, a central processing unit (CPU), an application specific integrated circuit (ASIC), a field-programmable gate array (FPGA), and/or another hardware device. Although a single processor 204 is depicted, it should be understood that the RS node 102 may include multiple processors, multiple cores, or the like, without departing from the scope of the RS node 102 system.

The RS node 102 may also include a non-transitory computer readable medium 212 that may have stored thereon machine-readable instructions executable by the processor 204. Examples of the machine-readable instructions are shown as 214-222 and are further discussed below. Examples of the non-transitory computer readable medium 212 may include an electronic, magnetic, optical, or other physical storage device that contains or stores executable instructions.

For example, the non-transitory computer readable medium 212 may be a Random-Access memory (RAM), an Electrically Erasable Programmable Read-Only Memory (EEPROM), a hard disk, an optical disc, or other type of storage device.

The processor 204 may fetch, decode, and execute the machine-readable instructions 214 to receive a care provider request 201 comprising user profile data from the at least one user device 101. The processor 204 may fetch, decode, and execute the machine-readable instructions 216 to parse the care provider request to derive a plurality of features from the user profile data. The processor 204 may fetch, decode, and execute the machine-readable instructions 218 to query an aggregated care providers' database 103 to retrieve historical care providers' matching data based on the user profile data. The processor 204 may fetch, decode, and execute the machine-readable instructions 220 to generate at least one feature vector based on the plurality of features and the historical care providers' matching data.

The processor 204 may fetch, decode, and execute the machine-readable instructions 222 to provide the at least one feature vector to the AI/ML module 107 for generating a predictive model 108 configured to output at least one care provider selection parameter for generating a care provider recommendation responsive to the care provider request. The permissioned blockchain 110 may be configured to use one or more smart contracts that manage transactions for multiple participating nodes and for recording the transactions on the ledger 109.

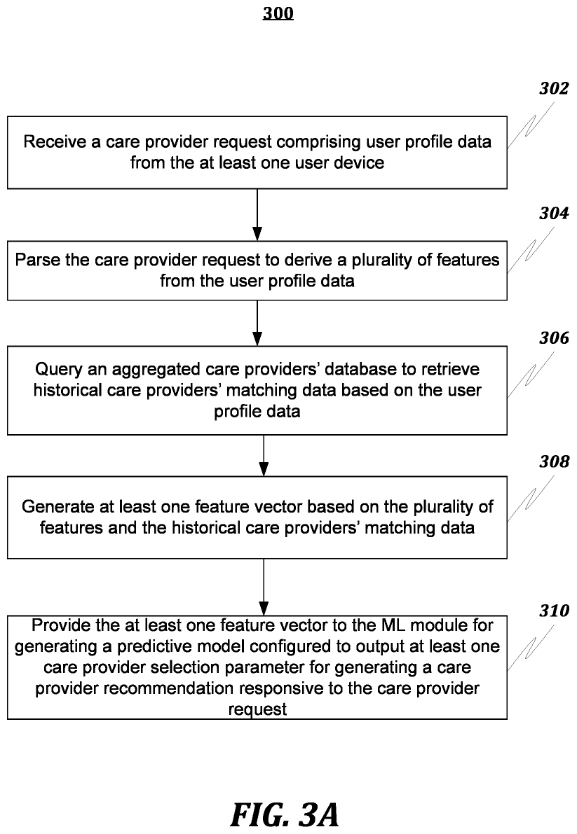

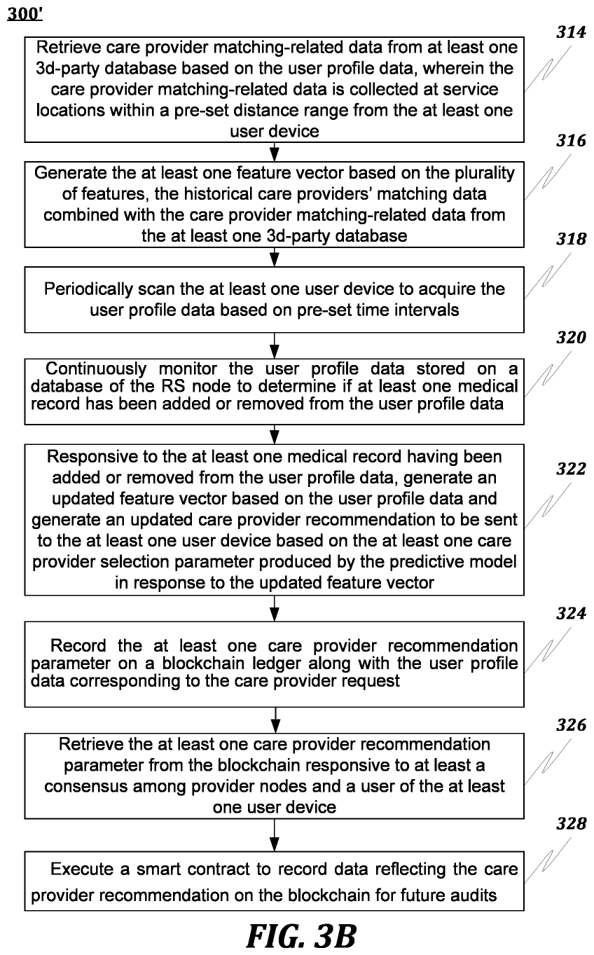

FIG. 3A illustrates a flowchart of a method for an intelligent AI-based automated care provider recommendation consistent with the present disclosure.

Referring to FIG. 3A, the method 300 may include one or more of the steps described below. FIG. 3A illustrates a flow chart of an example method executed by the RS node 102 (see FIG. 2). It should be understood that method 300 depicted in FIG. 3A may include additional operations and that some of the operations described therein may be removed and/or modified without departing from the scope of the method 300. The description of the method 300 is also made with reference to the features depicted in FIG. 2 for purposes of illustration. Particularly, the processor 204 of the RS node 102 may execute some or all of the operations included in the method 300.

With reference to FIG. 3A, at block 302, the processor 204 may receive a care provider request comprising user profile data from the at least one user device. At block 304, the processor 204 may parse the care provider request to derive a plurality of features from the user profile data. At block 306, the processor 204 may query an aggregated care providers' database to retrieve historical care providers' matching data based on the user profile data. At block 308, the processor 204 may generate at least one feature vector based on the plurality of features and the historical care providers' matching data. At block 310, the processor 204 may provide the at least one feature vector to the ML module for generating a predictive model configured to output at least one care provider selection parameter for generating a care provider recommendation responsive to the care provider request.

In one embodiment, the updated current care provider offer(s) data from the aggregated database 103 may be used for generation of the predictive model as well. If a medical record is added or removed from the user profile stored on the RS node 102 database, this change may be detected and the current care provided may be alerted. For example, if a user has been diagnosed with dementia, the care provider is immediately made aware of this diagnosis so the care for the user (i.e., patients) may be adjusted accordingly.

FIG. 3B illustrates a further flowchart of a method for an intelligent AI-based automated care provider recommendation consistent with the present disclosure. Referring to FIG. 3B, the method 300′ may include one or more of the steps described below. FIG. 3B illustrates a flow chart of an example method executed by the RS node 102 (see FIG. 2). It should be understood that method 300′ depicted in FIG. 3B may include additional operations and that some of the operations described therein may be removed and/or modified without departing from the scope of the method 300′. The description of the method 300′ is also made with reference to the features depicted in FIG. 2 for purposes of illustration. Particularly, the processor 204 of the RS node 102 may execute some or all of the operations included in the method 300′.

With reference to FIG. 3B, at block 314, the processor 204 may retrieve care provider matching-related data from at least one 3d-party database based on the user profile data, wherein the care provider matching-related data is collected at service locations within a pre-set distance range from the at least one user device. At block 316, the processor 204 may generate the at least one feature vector based on the plurality of features, the historical care providers' matching data combined with the care provider matching-related data from the at least one 3d-party database. At block 318, the processor 204 may periodically scan the at least one user device to acquire the user profile data based on pre-set time intervals. At block 320, the processor 204 may continuously monitor the user profile data stored on a database of the RS node 102 to determine if at least one medical record has been added or removed from the user profile data. If a new diagnosis or a notice form a doctor/nurse has been added, the RS node 102 determines this by scanning the database. Then, the current care provided is alerted of changes. In one embodiment a different specialized care provided may be recommended by the RS node 102.

In one embodiment, the processor 204 may, responsive to the at least one medical record having been added or removed from the user profile data, generate an updated feature vector based on the user profile data and generate an updated care provider recommendation to be sent to the at least one user device based on the at least one care provider selection parameter produced by the predictive model in response to the updated feature vector. The current care provider offers may be used in generation of an updated feature vector. At block 322, the processor 204 may record the at least one care provider recommendation parameter on a blockchain ledger along with the user profile data corresponding to the care provider request. At block 324, the processor 204 may retrieve the at least one care provider recommendation parameter from the blockchain responsive to at least a consensus among provider nodes and a user of the at least one user device. At block 326, the processor 204 may execute a smart contract to record data reflecting the care provider recommendation on the blockchain for future audits.

In one disclosed embodiment, the care provider recommendation parameters' model may be generated by the AI/ML module 107 that may use training data sets to improve accuracy of the prediction of the care provider recommendation parameters for the user of the device 101 (FIG. 2). The care provider recommendation parameters used in training data sets may be stored in a centralized local database (such as one used for storing aggregated data 103 depicted in FIG. 1A). In one embodiment, a neural network may be used in the AI/ML module 107 for care provider recommendation modeling and updating care provider recommendations.

In another embodiment, the AI/ML module 107 may use a decentralized storage such as a blockchain 110 (see FIG. 1B) that is a distributed storage system, which includes multiple nodes that communicate with each other. The decentralized storage includes an append-only immutable data structure resembling a distributed ledger capable of maintaining records between mutually untrusted parties. The untrusted parties are referred to herein as peers or peer nodes. Each peer maintains a copy of the parameter(s) records and no single peer can modify the records without a consensus being reached among the distributed peers. For example, the peers 113 and 102 (FIG. 1B) may execute a consensus protocol to validate blockchain storage transactions, group the storage transactions into blocks, and build a hash chain over the blocks. This process forms the ledger by ordering the storage transactions, as is necessary, for consistency. In various embodiments, a permissioned and/or a permissionless blockchain can be used. In a public or permissionless blockchain, anyone can participate without a specific identity. Public blockchains can involve assets and use consensus based on various protocols such as Proof of Work (PoW). On the other hand, a permissioned blockchain provides secure interactions among a group of entities which share a common goal such as storing card usage recommendation parameters for efficient usage of the payment cards, but which do not fully trust one another.

This application utilizes a permissioned (private) blockchain that operates arbitrary, programmable logic, tailored to a decentralized storage scheme and referred to as “smart contracts” or “chaincodes.” In some cases, specialized chaincodes may exist for management functions and parameters which are referred to as system chaincodes. The application can further utilize smart contracts that are trusted distributed applications which leverage tamper-proof properties of the blockchain database and an underlying agreement between nodes, which is referred to as an endorsement or endorsement policy. Blockchain transactions associated with this application can be “endorsed” before being committed to the blockchain while transactions, which are not endorsed, are disregarded. An endorsement policy allows chaincodes to specify endorsers for a transaction in the form of a set of peer nodes that are necessary for endorsement. When a client sends the transaction to the peers specified in the endorsement policy, the transaction is executed to validate the transaction. After a validation, the transactions enter an ordering phase in which a consensus protocol is used to produce an ordered sequence of endorsed transactions grouped into blocks.

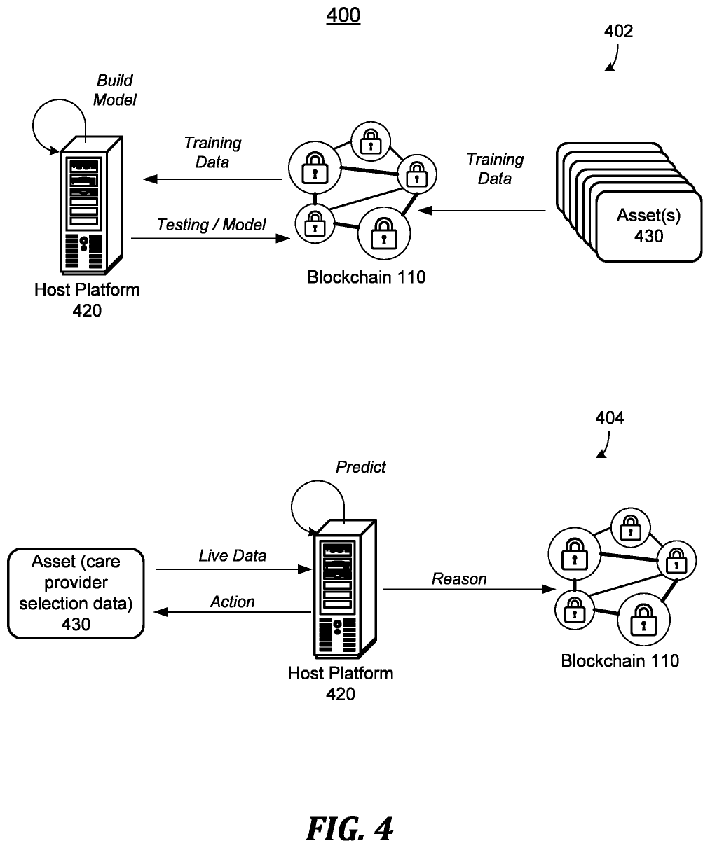

In the example depicted in FIG. 4, a host platform 420 (such as a RS node 102) builds and deploys a machine learning model for predictive monitoring of assets 430. Here, the host platform 420 may be a cloud platform, an industrial server, a web server, a personal computer, a user device, and the like. Assets 430 can represent care provider recommendation parameters. The blockchain 110 can be used to significantly improve both a training process 402 of the machine learning model and the care provider selection parameters' predictive process 405 based on a trained machine learning model. For example, in 402, rather than requiring a data scientist/engineer or other user to collect the data, historical data (heuristics—i.e., care provider matching-related data) may be stored by the assets 430 themselves (or through an intermediary, not shown) on the blockchain 110.

This can significantly reduce the collection time needed by the host platform 420 when performing predictive model training. For example, using smart contracts, data can be directly and reliably transferred straight from its place of origin (e.g., from the user device 101 or from the database 103) to the blockchain 110. By using the blockchain 110 to ensure the security and ownership of the collected data, smart contracts may directly send the data from the assets to the entities that use the data for building a machine learning model. This allows for sharing of data among the assets 430. The collected data may be stored in the blockchain 110 based on a consensus mechanism. The consensus mechanism pulls in (permissioned nodes 101, 102, 105 and 113) to ensure that the data being recorded is verified and accurate. The data recorded is timestamped, cryptographically signed, and immutable. It is therefore auditable, transparent, and secure.

Furthermore, training of the machine learning model on the collected data may take rounds of refinement and testing by the host platform 420. Each round may be based on additional data or data that was not previously considered to help expand the knowledge of the machine learning model. In 402, the different training and testing steps (and the data associated therewith) may be stored on the blockchain 110 by the host platform 420. Each refinement of the machine learning model (e.g., changes in variables, weights, etc.) may be stored on the blockchain 110. This provides verifiable proof of how the model was trained and what data was used to train the model. Furthermore, when the host platform 420 has achieved a finally trained model, the resulting model itself may be stored on the blockchain 110.

After the model has been trained, it may be deployed to a live environment where it can make optimal care provider selection predictions/decisions based on the execution of the final trained machine learning model using the care provider recommendation parameters. In this example, data fed back from the asset 430 may be input into the machine learning model and may be used to make event predictions such as most optimal care provider selection parameters for generation of care provider recommendation to the user. Determinations made by the execution of the machine learning model (e.g., care provider selection parameters, etc.) at the host platform 420 may be stored on the blockchain 110 to provide auditable/verifiable proof. As one non-limiting example, the machine learning model may predict a future change of a part of the asset 430 (the care provider selection parameters). The data behind this decision may be stored by the host platform 420 on the blockchain 110.

As discussed above, in one embodiment, the features and/or the actions described and/or depicted herein can occur on or with respect to the blockchain 110.

The above embodiments of the present disclosure may be implemented in hardware, in a computer-readable instructions executed by a processor, in firmware, or in a combination of the above. The computer computer-readable instructions may be embodied on a computer-readable medium, such as a storage medium. For example, the computer computer-readable instructions may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

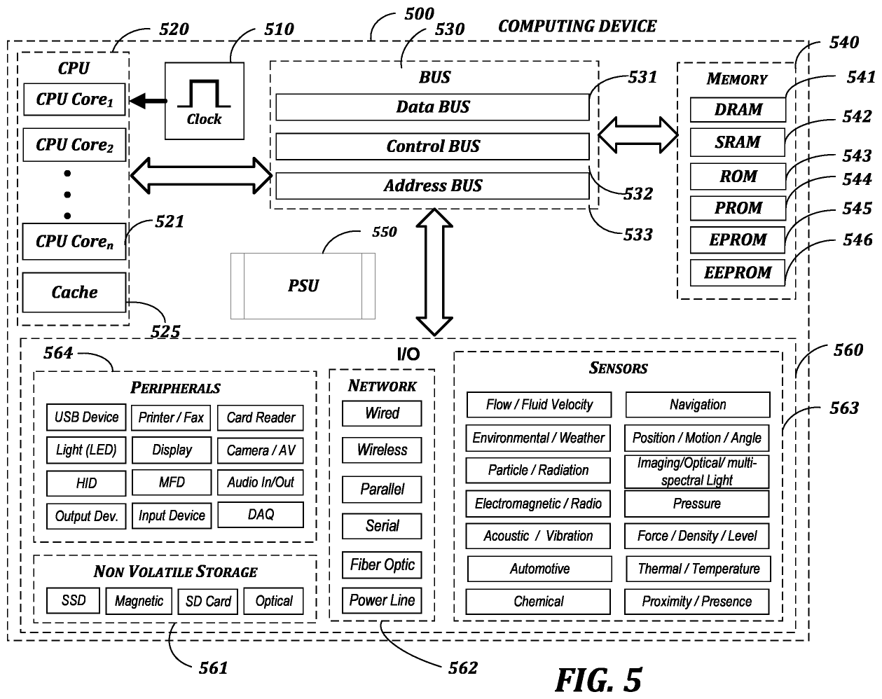

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative embodiment, the processor and the storage medium may reside as discrete components. For example, FIG. 5 illustrates an example computing device 500 (e.g., the RS node 102), which may represent or be integrated in any of the above-described components, etc.

FIG. 5 illustrates a block diagram of a system including computing device 500. The computing device 500 may comprise, but not be limited to the following:

Mobile computing device, such as, but is not limited to, a laptop, a tablet, a smartphone, a drone, a wearable, an embedded device, a handheld device, an Arduino, an industrial device, or a remotely operable recording device;

A supercomputer, an exa-scale supercomputer, a mainframe, or a quantum computer;

A minicomputer, wherein the minicomputer computing device comprises, but is not limited to, an IBM AS500/iSeries/System I, A DEC VAX/PDP, a HP3000, a Honeywell-Bull DPS, a Texas Instruments TI-990, or a Wang Laboratories VS Series;

A microcomputer, wherein the microcomputer computing device comprises, but is not limited to, a server, wherein a server may be rack mounted, a workstation, an industrial device, a raspberry pi, a desktop, or an embedded device;

The RS node 102 (see FIG. 2) may be hosted on a centralized server or on a cloud computing service. Although method 300 has been described to be performed by the RS node 102 implemented on a computing device 500, it should be understood that, in some embodiments, different operations may be performed by a plurality of the computing devices 500 in operative communication at least one network.

Embodiments of the present disclosure may comprise a computing device having a central processing unit (CPU) 520, a bus 530, a memory unit 550, a power supply unit (PSU) 550, and one or more Input/Output (I/O) units. The CPU 520 coupled to the memory unit 550 and the plurality of I/O units 560 via the bus 530, all of which are powered by the PSU 550. It should be understood that, in some embodiments, each disclosed unit may actually be a plurality of such units for the purposes of redundancy, high availability, and/or performance. The combination of the presently disclosed units is configured to perform the stages any method disclosed herein.

Consistent with an embodiment of the disclosure, the aforementioned CPU 520, the bus 530, the memory unit 550, a PSU 550, and the plurality of I/O units 560 may be implemented in a computing device, such as computing device 500. Any suitable combination of hardware, software, or firmware may be used to implement the aforementioned units. For example, the CPU 520, the bus 530, and the memory unit 550 may be implemented with computing device 500 or any of other computing devices 500, in combination with computing device 500. The aforementioned system, device, and components are examples and other systems, devices, and components may comprise the aforementioned CPU 520, the bus 530, the memory unit 550, consistent with embodiments of the disclosure.

At least one computing device 500 may be embodied as any of the computing elements illustrated in all of the attached figures, including the RS node 102 (FIG. 2). A computing device 500 does not need to be electronic, nor even have a CPU 520, nor bus 530, nor memory unit 550. The definition of the computing device 500 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 500, especially if the processing is purposeful.

With reference to FIG. 5, a system consistent with an embodiment of the disclosure may include a computing device, such as computing device 500. In a basic configuration, computing device 500 may include at least one clock module 510, at least one CPU 520, at least one bus 530, and at least one memory unit 550, at least one PSU 550, and at least one I/O 560 module, wherein I/O module may be comprised of, but not limited to a non-volatile storage sub-module 561, a communication sub-module 562, a sensors sub-module 563, and a peripherals sub-module 565.

A system consistent with an embodiment of the disclosure the computing device 500 may include the clock module 510 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 520, the central component of modern computers, which relies on a clock. The only exceptions are asynchronous circuits such as asynchronous CPUs. The clock 510 can comprise a plurality of embodiments, such as, but not limited to, single-phase clock which transmits all clock signals on effectively 1 wire, two-phase clock which distributes clock signals on two wires, each with nonoverlapping pulses, and four-phase clock which distributes clock signals on 5 wires.

Many computing devices 500 use a “clock multiplier” which multiplies a lower frequency external clock to the appropriate clock rate of the CPU 520. This allows the CPU 520 to operate at a much higher frequency than the rest of the computer, which affords performance gains in situations where the CPU 520 does not need to wait on an external factor (like memory 550 or input/output 560). Some embodiments of the clock 510 may include dynamic frequency change, where, the time between clock edges can vary widely from one edge to the next and back again.

A system consistent with an embodiment of the disclosure the computing device 500 may include the CPU unit 520 comprising at least one CPU Core 521. A plurality of CPU cores 521 may comprise identical CPU cores 521, such as, but not limited to, homogeneous multi-core systems. It is also possible for the plurality of CPU cores 521 to comprise different CPU cores 521, such as, but not limited to, heterogeneous multi-core systems, big.LITTLE systems and some AMD accelerated processing units (APU). The CPU unit 520 reads and executes program instructions which may be used across many application domains, for example, but not limited to, general purpose computing, embedded computing, network computing, digital signal processing (DSP), and graphics processing (GPU). The CPU unit 520 may run multiple instructions on separate CPU cores 521 at the same time. The CPU unit 520 may be integrated into at least one of a single integrated circuit die and multiple dies in a single chip package. The single integrated circuit die and multiple dies in a single chip package may contain a plurality of other aspects of the computing device 500, for example, but not limited to, the clock 510, the CPU 520, the bus 530, the memory 550, and I/O 560.

The CPU unit 520 may contain cache 522 such as, but not limited to, a level 1 cache, level 2 cache, level 3 cache or combination thereof. The aforementioned cache 522 may or may not be shared amongst a plurality of CPU cores 521. The cache 522 sharing comprises at least one of message passing and inter-core communication methods may be used for the at least one CPU Core 521 to communicate with the cache 522. The inter-core communication methods may comprise, but not limited to, bus, ring, two-dimensional mesh, and crossbar. The aforementioned CPU unit 520 may employ symmetric multiprocessing (SMP) design.

The plurality of the aforementioned CPU cores 521 may comprise soft microprocessor cores on a single field programmable gate array (FPGA), such as semiconductor intellectual property cores (IP Core). The plurality of CPU cores 521 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 521, for example, but not limited to Instruction-level parallelism (ILP) such as, but not limited to, superscalar pipelining, and Thread-level parallelism (TLP).

Consistent with the embodiments of the present disclosure, the aforementioned computing device 500 may employ a communication system that transfers data between components inside the aforementioned computing device 500, and/or the plurality of computing devices 500. The aforementioned communication system will be known to a person having ordinary skill in the art as a bus 530. The bus 530 may embody internal and/or external plurality of hardware and software components, for example, but not limited to a wire, optical fiber, communication protocols, and any physical arrangement that provides the same logical function as a parallel electrical bus. The bus 530 may comprise at least one of, but not limited to a parallel bus, wherein the parallel bus carry data words in parallel on multiple wires, and a serial bus, wherein the serial bus carry data in bit-serial form. The bus 530 may embody a plurality of topologies, for example, but not limited to, a multidrop/electrical parallel topology, a daisy chain topology, and a connected by switched hubs, such as USB bus. The bus 530 may comprise a plurality of embodiments, for example, but not limited to:
Internal data bus (data bus) 531/Memory bus
Control bus 532
Address bus 533
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
l-Wire
Peripheral Component Interconnect (PCI), including embodiments such as, but not limited to, Accelerated Graphics Port (AGP), Peripheral Component Interconnect eXtended (PCIX), Peripheral Component Interconnect Express (PCI-e) (e.g., PCI Express Mini Card, PCI Express M.2 [Mini PCIe v2], PCI Express External Cabling [ePCIe], and PCI Express OCuLink [Optical Copper{Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/105 bus (e.g., PC/105-Plus, PCI/105-Express, PCI/105, and PCI-105), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1395 Interface/Firewire, Thunderbolt, and eXtensible Host Controller Interface (xHCI).

Consistent with the embodiments of the present disclosure, the aforementioned computing device 500 may employ hardware integrated circuits that store information for immediate use in the computing device 500, know to the person having ordinary skill in the art as primary storage or memory 550. The memory 550 operates at high speed, distinguishing it from the non-volatile storage sub-module 561, which may be referred to as secondary or tertiary storage, which provides slow-to-access information but offers higher capacities at lower cost. The contents contained in memory 550, may be transferred to secondary storage via techniques such as, but not limited to, virtual memory and swap. The memory 550 may be associated with addressable semiconductor memory, such as integrated circuits consisting of silicon-based transistors, used for example as primary storage but also other purposes in the computing device 500. The memory 550 may comprise a plurality of embodiments, such as, but not limited to volatile memory, non-volatile memory, and semi-volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 551, Static Random-Access Memory (SRAM) 552, CPU Cache memory 525, Advanced Random-Access Memory (A-RAM), and other types of primary storage such as Random-Access Memory (RAM).
Non-volatile memory which can retain stored information even after power is removed, for example, but not limited to, Read-Only Memory (ROM) 553, Programmable ROM (PROM) 555, Erasable PROM (EPROM) 555, Electrically Erasable PROM (EEPROM) 556 (e.g., flash memory and Electrically Alterable PROM [EAPROM]), Mask ROM (MROM), One Time Programable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).
Consistent with the embodiments of the present disclosure, the aforementioned computing device 500 may employ the communication system between an information processing system, such as the computing device 500, and the outside world, for example, but not limited to, human, environment, and another computing device 500. The aforementioned communication system will be known to a person having ordinary skill in the art as I/O 560. The I/O module 560 regulates a plurality of inputs and outputs with regard to the computing device 500, wherein the inputs are a plurality of signals and data received by the computing device 500, and the outputs are the plurality of signals and data sent from the computing device 500. The I/O module 560 interfaces a plurality of hardware, such as, but not limited to, non-volatile storage 561, communication devices 562, sensors 563, and peripherals 565. The plurality of hardware is used by the at least one of, but not limited to, human, environment, and another computing device 500 to communicate with the present computing device 500. The I/O module 560 may comprise a plurality of forms, for example, but not limited to channel I/O, port mapped I/O, asynchronous I/O, and Direct Memory Access (DMA).
Consistent with the embodiments of the present disclosure, the aforementioned computing device 500 may employ the non-volatile storage sub-module 561, which may be referred to by a person having ordinary skill in the art as one of secondary storage, external memory, tertiary storage, off-line storage, and auxiliary storage. The non-volatile storage sub-module 561 may not be accessed directly by the CPU 520 without using intermediate area in the memory 550. The non-volatile storage sub-module 561 does not lose data when power is removed and may be two orders of magnitude less costly than storage used in memory module, at the expense of speed and latency. The non-volatile storage sub-module 561 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (561) may comprise a plurality of embodiments, such as, but not limited to:
Optical storage, for example, but not limited to, Compact Disk (CD) (CD-ROM/CDR/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the aforementioned computing device 500 may employ the communication sub-module 562 as a subset of the I/O 560, which may be referred to by a person having ordinary skill in the art as at least one of, but not limited to, computer network, data network, and network. The network allows computing devices 500 to exchange data using connections, which may be known to a person having ordinary skill in the art as data links, between network nodes. The nodes comprise network computer devices 500 that originate, route, and terminate data. The nodes are identified by network addresses and can include a plurality of hosts consistent with the embodiments of a computing device 500. The aforementioned embodiments include, but not limited to personal computers, phones, servers, drones, and networking devices such as, but not limited to, hubs, switches, routers, modems, and firewalls.

Two nodes can be said are networked together, when one computing device 500 is able to exchange information with the other computing device 500, whether or not they have a direct connection with each other. The communication sub-module 562 supports a plurality of applications and services, such as, but not limited to World Wide Web (WWW), digital video and audio, shared use of application and storage computing devices 500, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 5 [IPv5], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [DEN]).

The communication sub-module 562 may comprise a plurality of size, topology, traffic control mechanism and organizational intent. The communication sub-module 562 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Wherein cellular systems embody technologies such as, but not limited to, 3G, 5G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line and wireless communications

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 500 may employ the sensors sub-module 563 as a subset of the I/O 560. The sensors sub-module 563 comprises at least one of the devices, modules, and subsystems whose purpose is to detect events or changes in its environment and send the information to the computing device 500. Sensors are sensitive to the measured property, are not sensitive to any property not measured, but may be encountered in its application, and do not significantly influence the measured property. The sensors sub-module 563 may comprise a plurality of digital devices and analog devices, wherein if an analog device is used, an Analog to Digital (A-to-D) converter must be employed to interface the said device with the computing device 500. The sensors may be subject to a plurality of deviations that limit sensor accuracy. The sensors sub-module 563 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:

Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nanosensors).

Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (02), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermoluminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, LiDAR, multi-spectral light sensor, colorimeter, contact image sensor, electro-optical sensor, infrared sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photoswitch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating Utube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezocapacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 500 may employ the peripherals sub-module 562 as a subset of the I/O 560. The peripheral sub-module 565 comprises ancillary devices uses to put information into and get information out of the computing device 500. There are 3 categories of devices comprising the peripheral sub-module 565, which exist based on their relationship with the computing device 500, input devices, output devices, and input/output devices. Input devices send at least one of data and instructions to the computing device 500. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.
The number of degrees of freedom involved, such as, but not limited to, two-dimensional mice vs three-dimensional mice used for Computer-Aided Design (CAD) applications.

Output devices provide output from the computing device 500. Output devices convert electronically generated information into a form that can be presented to humans. Input/output devices perform that perform both input and output functions. It should be understood by a person having ordinary skill in the art that the ensuing are non-limiting embodiments of the aforementioned peripheral sub-module 565:

Input Devices
Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).
High degree of freedom devices, that require up to six degrees of freedom such as, but not limited to, camera gimbals, Cave Automatic Virtual Environment (CAVE), and virtual reality systems.
Video Input devices are used to digitize images or video from the outside world into the computing device 500. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
Audio input devices are used to capture sound. In some cases, an audio output device can be used as an input device, in order to capture produced sound. Audio input devices allow a user to send audio signals to the computing device 500 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrumental Digital Interface (MIDI) devices such as, but not limited to a keyboard, and headset.
Data Acquisition (DAQ) devices convert at least one of analog signals and physical parameters to digital values for processing by the computing device 500. Examples of DAQ devices may include, but not limited to, Analog to Digital Converter (ADC), data logger, signal conditioning circuitry, multiplexer, and Time to Digital Converter (TDC).

Output Devices may further comprise, but not be limited to:
Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).

Printers, such as, but not limited to, inkjet printers, laser printers, 3D printers, solid ink printers and plotters.
Audio and Video (AV) devices, such as, but not limited to, speakers, headphones, amplifiers and lights, which include lamps, strobes, DJ lighting, stage lighting, architectural lighting, special effect lighting, and lasers.
Other devices such as Digital to Analog Converter (DAC)

Input/Output Devices may further comprise, but not be limited to, touchscreens, networking device (e.g., devices disclosed in network 562 sub-module), data storage device (non-volatile storage 561), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

What is claimed is:

1. A system, comprising:
a processor of a recommendation server (RS) node connected to at least one user device over a network and configured to host a machine learning (ML) module;
a memory on which are stored machine-readable instructions that when executed by the processor, cause the processor to:
receive a care provider request comprising user profile data from the at least one user device;
parse the care provider request to derive a plurality of features from the user profile data;
query an aggregated care providers' database to retrieve historical care providers' matching data based on the user profile data;
generate at least one feature vector based on the plurality of features and the historical care providers' matching data; and
provide the at least one feature vector to the ML module for generating a predictive model configured to output at least one care provider selection parameter for generating a care provider recommendation responsive to the care provider request.

2. The system of claim 1, wherein the instructions further cause the processor to retrieve care provider matching-related data from at least one 3d-party database based on the user profile data, wherein the care provider matching-related data is collected at service locations within a pre-set distance range from the at least one user device.

3. The system of claim 2, wherein the instructions further cause the processor to generate the at least one feature vector based on the plurality of features, the historical care providers' matching data combined with the care provider matching-related data from the at least one 3d-party database.

4. The system of claim 1, wherein the instructions further cause the processor to periodically scan the at least one user device to acquire the user profile data based on pre-set time intervals.

5. The system of claim 1, wherein the instructions further cause the processor to continuously monitor the user profile data stored on a database of the RS node to determine if at least one medical record has been added or removed from the user profile data.

6. The system of claim 5, wherein the instructions further cause the processor to, responsive to the at least one medical record having been added or removed from the user profile data, generate an updated feature vector based on the user profile data and generate an updated care provider recommendation to be sent to the at least one user device based on the at least one care provider selection parameter produced by the predictive model in response to the updated feature vector.

7. The system of claim 1, wherein the instructions further cause the processor to record the at least one care provider recommendation parameter on a blockchain ledger along with the user profile data corresponding to the care provider request.

8. The system of claim 7, wherein the instructions further cause the processor to retrieve the at least one care provider recommendation parameter from the blockchain responsive to at least a consensus among provider nodes and a user of the at least one user device.

9. The system of claim 8, wherein the instructions further cause the processor to execute a smart contract to record data reflecting the care provider recommendation on the blockchain for future audits.

10. A method for care provider recommendation, comprising:
receiving, by a recommendation server (RS) node, a care provider request comprising user profile data from at least one user device;
parsing, by the RS node, the care provider request to derive a plurality of features from the user profile data;
querying, by the RS node, an aggregated care providers' database to retrieve historical care providers' matching data based on the user profile data;
generating, by the RS node, at least one feature vector based on the plurality of features and the historical care providers' matching data; and
providing, by the RS node, the at least one feature vector to the ML module for generating a predictive model configured to output at least one care provider selection parameter for generating a care provider recommendation responsive to the care provider request.

11. The method of claim 10, further comprising retrieving care provider matching-related data from at least one 3d-party database based on the user profile data, wherein the care provider matching-related data is collected at service locations within a pre-set distance range from the at least one user device.

12. The method of claim 11, further comprising generating the at least one feature vector based on the plurality of features, the historical care providers' matching data combined with the care provider matching-related data from the at least one 3d-party database.

13. The method of claim 10, further comprising periodically scanning the at least one user device to acquire the user profile data based on pre-set time intervals.

14. The method of claim 10, further comprising continuously monitoring the user profile data stored on a database of the RS node to determine if at least one medical record has been added or removed from the user profile data.

15. The method of claim 14, further comprising, responsive to the at least one medical record having been added or removed from the user profile data, generating an updated feature vector based on the user profile data and generating an updated care provider recommendation to be sent to the at least one user device based on the at least one care provider selection parameter produced by the predictive model in response to the updated feature vector.

16. A non-transitory computer readable medium comprising instructions, that when read by a processor, cause the processor to perform:
receiving a care provider request comprising user profile data from at least one user device;
parsing the care provider request to derive a plurality of features from the user profile data;
querying an aggregated care providers' database to retrieve historical care providers' matching data based on the user profile data;
generating at least one feature vector based on the plurality of features and the historical care providers' matching data; and
providing the at least one feature vector to the ML module for generating a predictive model configured to output at least one care provider selection parameter for generating a care provider recommendation responsive to the care provider request.

17. The non-transitory computer readable medium of claim 16, further comprising instructions, that when read by the processor, cause the processor to retrieve care provider matching-related data from at least one 3d-party database based on the user profile data, wherein the care provider matching-related data is collected at service locations within a pre-set distance range from the at least one user device.

18. The non-transitory computer readable medium of claim 16, further comprising instructions, that when read by the processor, cause the processor to generate the at least one feature vector based on the plurality of features, the historical care providers' matching data combined with the care provider matching-related data from the at least one 3d-party database.

19. The non-transitory computer readable medium of claim 16, further comprising instructions, that when read by the processor, cause the processor to periodically scan the at least one user device to acquire the user profile data based on pre-set time intervals.

20. The non-transitory computer readable medium of claim 19, further comprising instructions, that when read by the processor, cause the processor to continuously monitor the user profile data stored on a database of the RS node to determine if at least one medical record has been added or removed from the user profile data and, responsive to the at least one medical record having been added or removed from the user profile data, alerting a care provider associated with the care provider recommendation.
